(19) United States
(12) Patent Application Publication
(10) Pub. No.: US 2001/0056066 A1
(43) Pub. Date: Dec. 27, 2001

(54) Method of treating immune cell mediated systemic diseases

(75) Inventors: Peter John Bugelski, Drexel Hill, PA (US); Charles Baldwin Davis, Devon, PA (US); Brian Richard MacDonald, Valley Forge, PA (US)

Correspondence Address: GLAXOSMITHKLINE, Corporate Intellectual Property - UW2220, P.O. Box 1539, King of Prussia, PA, 19406-0939, US

(73) Assignee: SmithKline Beecham Corporation

(21) Appl. No.: 09/905,836

(22) Filed: Jul. 13, 2001

Related U.S. Application Data
(63) Continuation of application No. 09/230,119, filed on May 26, 1999, now abandoned.
Application No. 09/230,119 is a 371 of international application No. PCT/US97/12600, filed on Jul. 25, 1997.
(60) Provisional application No. 60/022,472, filed on Jul. 26, 1996.

(51) Int. Cl. A61K 38/16; A61K 39/395

(52) U.S. Cl. 514/8; 424/131.1

(57) ABSTRACT

An improved method of treating immune cell mediated systemic diseases, particularly T and B cell mediated diseases, is provided by increasing the systemic exposure, or bioavailibility, of a therapeutic protein. Such therapeutic protein is selected from the group consisting of a monoclonal antibody, a soluble receptor and a soluble ligand which binds to an antigen expressed on the surface of an immunce cell.

### FIELD OF INVENTION

The present invention relates generally to the field of monoclonal antibodies, routes of administration, and treatment of immune cell mediated diseases.

### BACKGROUND

Currently, there are numerous monoclonal antibodies in clinical testing or development for a variety of in vivo uses such as fertility testing, diagnosis of sepsis, therapeutic applications such as for organ transplantation, treatment of autoimmune disease, restenosis, certain forms of cancer, as well as prophylactic applications, e.g., as an anti-viral agent. Typically such antibodies are administered either intravenously (iv) or subcutaneously (sc), although other routes of administration are also possible, e.g., intramuscularly (im) and intranasally. In general, sc administration is preferable over iv administration, for iv administration requires catheterization for administration in a home setting, medical attention when administered in a clinic or physician's office, or hospitalization in more extreme circumstances. In addition, a therapeutic delivered iv takes longer to administer when compared to sc administration, and as a result is a more costly therapy. However, sc administration is not without drawbacks. For example, there are physical limitations on the maximum dose which can be delivered at the injection site.

It has also been observed that large polypeptides, such as antibodies, when administered subcutaneously, are first absorbed into the lymphatic system from the site of injection and then subsequently migrate into the blood stream (see, e.g., Weinstein et al., Science, 222: 423-426 (1983), Weinstein et al., Cancer Invest., 3:85-95 (1985), Supersaxo et al., Pharm. Res., 7:167-169 (1990)). For antigen targets not located in the lymphatic system, e.g., respiratory syncytial virus, the systemic exposure of the antibody administered sc is comparable to that administered iv (Davis et al., Drug Met. Disp., 23:1028-1036 (1995)).

However, Applicants have discovered that when an antibody targets or binds an antigen expressed on the surface of immune cells, e.g., T (or B) cells, the extent of absorption (i.e., systemic exposure) is limited by binding of antibody to such antigen in the lymphatic system, thus preventing antibody from entering the blood stream. Thus, to treat systemic immune cell mediated diseases, such as rheumatoid arthritis, it is desirable for the antibody to reach the ultimate site(s) of action in an effective amount. For a subcutaneous route of administration, it is essential for the antibody to enter the blood stream and not remain in the lymph nodes or other regions of the lymphatic system. Hence, the need exists to effectively deliver antibodies, and other therapeutic proteins, to treat systemic immune cell mediated (e.g., T or B-cell mediated) diseases. The methods described herein will become apparent to those of ordinary skill in the art upon reading this specification.

### SUMMARY OF INVENTION

This invention provides an improved method for treating immune cell mediated diseases by increasing the systemic exposure of therapeutic proteins which bind to selected antigens on the surface of immune cells. The systemic exposure of such therapeutic protein is increased by first providing (or administering) a saturating dose of the therapeutic protein followed by a second administration of such therapeutic protein, which is given subcutaneously, whereby the systemic exposure of the second administration is at least 50% greater than an equivalent subcutaneous dose administered without the benefit of the saturating dose. Preferably, the systemic exposure of such therapeutic protein is increased by at least 2-fold, more preferably it is increased by at least 4-fold.

In a preferred embodiment, the immune cell is a T (cell) lymphocyte.

In another preferred embodiment, the immune cell is a B (cell) lymphocyte.

### BRIEF DESCRIPTION OF DRAWINGS

FIG. 1 illustrates plasma radioactivity, as percent administered dose, following iv administration (0.4 mg/kg) of [³H]CE9.1 to CD4+ (circles) and CD4− (squares) transgenic mice. Open symbols represent total radioactivity while closed symbols represent radioactivity that bound Sepharose-conjugated soluble CD4. One male mouse was employed for each time point except for 24 and 48 hr, where 2 animals were used (mean percent shown for 24 and 48 h).

FIG. 2 illustrates total radioactivity, as percent administered dose, in spleen (circles) and thymus (squares) following iv administration (0.4 mg/kg) of [³H]CE9.1 to CD4+ (closed symbols) and CD4− (open symbols) transgenic mice. Spleen radioactivity in the CD4+ mice approached 20% of the administered dose in 2 hr while no uptake was observed in CD4− mice, or the thymus of CD4+ or CD4− mice.

FIG. 3 illustrates total radioactivity, as percent administered dose, in liver (circles). kidney (squares) and lung (triangles) following iv administration (0.4 mg/kg) of [³H]CE9.1 to CD4+ (closed symbols) and CD4− (open symbols) transgenic mice. By comparison with the spleen, CD4 receptor mediated uptake in liver, kidney and lung were not significant.

FIG. 4 illustrates dose dependence of percent administered radioactivity in spleen (A) and liver (B) after iv administration of [³H]CE9.1 to CD4+ or CD4− transgenic mice. The circles represent doses of 0.4 mg/kg while the squares represent doses of 100 mg/kg. The filled symbols represent data from knockouts (CD4−) while the open symbols represent data from CD4+ transgenic mice. As the dose was increased, liver radioactivity increased proportionately while spleen radioactivity did not. The radiolabel profile in the spleen of CD4+ mice at the high dose resembled the profile of CD4− mice at the low dose.

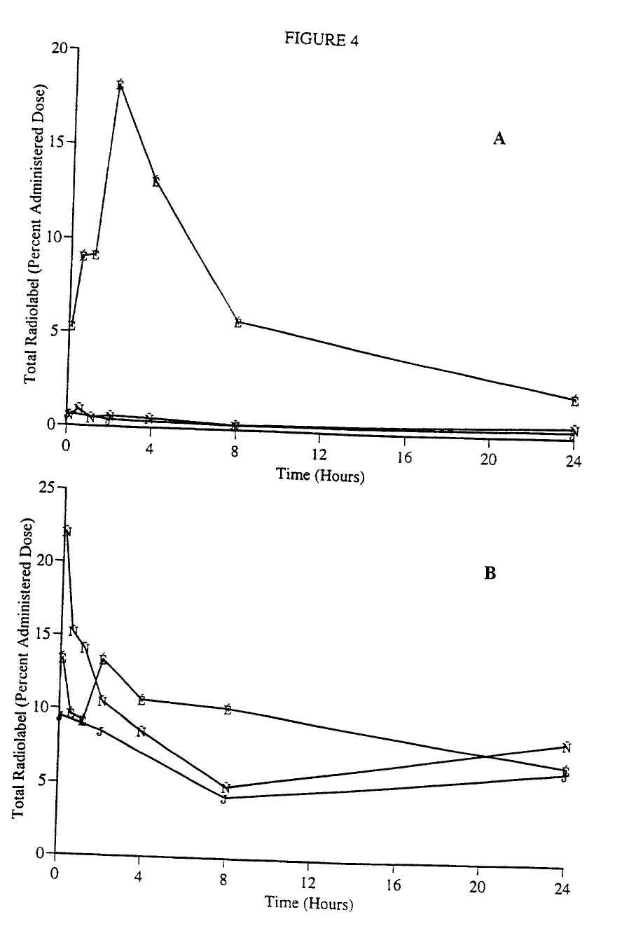

FIG. 5 illustrates plasma radioactivity, as percent administered dose, following sc administration (0.4 mg/kg) of [³H]CE9.1 to CD4+ (circles) and CD4− (squares) transgenic mice. Open symbols represent total radioactivity while closed symbols represent radioactivity that bound Sepharose-conjugated soluble CD4. One animal was employed for each time point. Though biologically active anti-CD4 mAb persisted in plasma of CD4− mice for weeks, in animals bearing the human receptor, no active mAb was observed at all after sc administration (plasma CE9.1 concentrations were non-quantifiable throughout, LLQ=10 ng/ml).

### DETAILED DESCRIPTION OF THE INVENTION

The present invention relates to an improved method for treating systemic, immune cell mediated diseases, such as autoimmune diseases, with a therapeutic protein that recognizes an antigen expressed on the surface of an immune cell. Applicants have found that by binding the immune cell antigen with a saturating amount of therapeutic protein, such as a monoclonal antibody, or other binding proteins, such as a soluble receptor (or a soluble ligand), then subsequent subcutaneous administration of the therapeutic protein results in systemic exposure which is increased, preferably at least 2-fold, relative to that observed from a single and equivalent sc dose alone.

### Definitions

An “immune cell” refers to those cells critical for immune response in an individual and which are commonly found in the lymphatic system, and in particular, in lymph nodes. Such cells include T cells (or T lymphocytes), B cells (or B lymphocytes), macrophages and dendritic cells.

“A saturating dose” refers to the amount of therapeutic protein necessary to completely bind a selected immune cell antigen in the lymphatic system such that no appreciable binding of the therapeutic protein to the immune cell antigen occurs upon subsequent administration(s) of the therapeutic protein. The amount of therapeutic protein needed will vary according to the amount of immune cell antigen present in the lymphatic system, the affinity of the therapeutic protein for such antigen, and the half-life of the therapeutic protein in vivo. One skilled in the art will be able to identify the appropriate amount of the therapeutic protein. For example, for a human anti-CD4 monoclonal antibody, the saturating dose will typically be in the range of 0.5 to 5 mg/kg.

“No appreciable binding” means that the difference between the plasma AUC (area under the plasma concentration versus time curve) between a subcutaneous dose which follows the saturating dose and a subcutaneous dose (of the same dosage) that was not preceded by a saturating dose, is at least 2-fold.

“A selected T (or B) cell antigen” refers to cell surface proteins expressed on T or B lymphocytes. Such proteins are typically receptors, or alternatively ligands to the receptors (also referred to as counter receptors), which are involved with T (or B) cell mediated disorders. Such antigens include, but are not limited to, CD3, CD4, CD8, CD11, CD18, CD20, CD23, CD28, gp39 (also known as CD40 ligand, CD40 counter receptor or T-BAM), CD40, CD80, CD86 as well as other members of the CD family.

“A therapeutic protein” can be a monoclonal antibody, or other protein that binds the selected immune cell ‘target’ antigen. Such protein is able to compete with the natural ligand of said antigen, or otherwise inhibit the interactions between immune cells, such as the interaction between T and B cells. The therapeutic protein can be a monoclonal antibody or other binding protein, such as a soluble receptor or soluble ligand (i.e., a soluble counter receptor). The soluble receptor (or counter receptor) comprises a protein wherein the transmembrane and/or the cytoplasmic regions have been deleted. Optionally, the soluble receptor (or counter receptor) can be fused to another protein to enhance or create desired properties. For example, the soluble receptor (or counter receptor) can be fused to an immunoglobulin Fc region to increase circulating half-life in vivo.

“The systemic exposure of a therapeutic protein” is the level of therapeutic protein in the bloodstream (bioavailability) as measured by the AUC.

### Features

The present invention relates to an improved method for treating systemic, immune cell mediated diseases, such as autoimmune diseases, with a therapeutic protein that recognizes an antigen expressed on the surface of the immune cell. It is based on the observation that systemic exposure of a therapeutic protein which binds to an immune cell antigen is highly dependent on the route of administration as well as the presence and distribution of such antigen in the lymphatic system. Applicants have made the unexpected discovery that when the first or initial dose is given subcutaneously, such therapeutic protein is not detected in the systemic circulation (bloodstream) unless given at a very high dose, presumably because the antigen and therapeutic protein complex in the lymphatic system thus preventing migration into the bloodstream.

By administering a therapeutic protein according to the present invention, the systemic exposure (i.e., plasma AUC) of the subcutaneous dose increases dramatically. That is, it can increase by a factor of 2 or more (100% or more). Preferably the systemic exposure from a subcutaneous dose increases by a factor of 4 or more. More preferably it increases by a factor of 10 or more. Thus, the method of treating immune cell disorders is improved by increasing the systemic exposure of the therapeutic protein.

Preferably, the therapeutic protein is a monoclonal antibody, although it is not limited to such. More preferably, the antibody is a human monoclonal antibody (see, e.g., WO 94/06448 “Human Neutralizing Monoclonal Antibodies to RSV”; Barbas et al., Methods a Companion to Methods in Enzymol., 2(2): 119-124 (1991) and; Burton et al., Proc. Nat'l. Acad. Sci, 88:10134-10137 (1991)) or an altered antibody which is a protein encoded by an altered immunoglobulin coding region, and expressed in a selected host cell. Such altered antibodies can either be engineered antibodies (e.g., chimeric or humanized antibodies) or antibody fragments lacking all or part of an immunoglobulin constant region, e.g., Fv, Fab, F(ab)₂ and the like.

For repeated or chronic dosing, it is preferable that the monoclonal antibody is either human or an engineered antibody. Such engineered antibody comprises a full-length synthetic antibody (e.g., a chimeric or humanized antibody, as opposed to an antibody fragment) in which a portion of the light and/or heavy chain variable domains of a selected acceptor antibody are replaced by analogous parts from one or more donor antibodies which have specificity for the selected epitope of an immune cell antigen. For example, such molecules may include antibodies characterized by a humanized heavy chain associated with an unmodified light chain (or chimeric light chain), or vice versa. Engineered antibodies may also be characterized by alteration of the nucleic acid sequences encoding the acceptor antibody light and/or heavy variable domain framework regions in order to retain donor antibody binding specificity. These antibodies can comprise replacement of one or more CDRs (preferably all) from the acceptor antibody with CDRs from a donor antibody described herein. Techniques for constructing engineered antibodies are known in the art (see, e.g., Queen et al., Proc. Natl Acad Sci USA, 86:10029-10032 (1989), Hodgson et al., Bio/Technology. 9:421 (1991)).

Preferably, the monoclonal antibody administered according to the invention recognizes a selected T (or B) cell antigen. One preferred embodiment is an antibody to the human CD4 receptor protein (antigen) on T lymphocytes. Such antibody can be chimeric, human or humanized in order to minimize antigenicity. For illustrative purposes, the human anti-CD4 antibody is primatized™ antibody, such as CE9.1 (see WO93/02108 which describes primate-human chimeric antibodies, i.e., primatizedtm, and Newman et al., Bio/Technology 10, 1455-1460 (1992) which describes CE9.1). Other preferred antigens include CD3, CD8, CD11, CD18, CD20, CD28, gp39 (also known as CD40 ligand), CD40, CD80 and CD86.

### Route of Administration

In general, subcutaneous administration is more desirable for doctors and patients than intravenous administration. Sc administration can be accomplished in minutes, rather than hours for iv infusion. Moreover, iv infusion is either administered: (i) in hospitals; (ii) or physician's offices; (iii) or in a patient's home, with catheter, whereas sc administration can be performed practically anywhere without catheterization.

Sc administration is also advantageous in that it typically avoids pain and bruising that often accompanies intramuscular (im) injection. This can be a significant advantage for persons who receive a therapeutic on a daily, or other frequent interval (e.g., weekly, bi-weekly, etc.). It is also postulated that sc administration can result in a longer circulating half-life for therapeutic proteins when compared to iv administration. For example, a soluble form of the CD4 receptor protein sCD4 (or sT4, see Deen et al., Nature, 331:82-84 (1988)) has a circulating half-life of approximately 6 minutes when administered iv, compared to approximately 1 hour when administered sc.

A disadvantage of subcutaneous dosing is the amount of therapeutic protein that can be administered. That is, it is not always feasible to deliver therapeutic proteins by a sc route of administration for there are limits on the total amount of therapeutic protein which can be given. This, in turn, is determined by the amount of solution which can be administered to a patient and the solubility of the therapeutic agent. As a general rule, the upper volume of solution which can be administered is approximately 2 ml. Preferably the volume would not exceed 1.5 ml, more preferably it would not exceed 1.3 ml.

Another disadvantage of subcutaneous dosing is that some therapeutic proteins are not stable in the lymph. That is, in some circumstances degradation may occur following subcutaneous administration.

However, the binding of an immune cell antigen, such as a T (or B) cell antigen, with a saturating amount of therapeutic protein such as a monoclonal antibody, allows subsequent subcutaneous administration(s) to achieve increased systemic exposure(s) of the therapeutic protein, preferably the increase is at least 2-fold relative to that observed from a single and equivalent (first) sc dose. For systemic diseases, such as rheumatoid arthritis, it is beneficial for the antibody to enter the systemic circulation without appreciable loss (and degradation) when passing through the lymphatic system. The present invention helps to facilitate this event, thus making a subcutaneous route of administration a more clinically feasible alternative.

Thus, an advantage of the present invention is that one can get comparable systemic exposure of a therapeutic protein without chronic iv dosing. Furthermore, the present invention allows a more convenient regimen for chronic administration of a therapeutic protein (e.g., mAb) and thus a more practical course of treatment. Another feature is that by providing an initial dose whereby immune cell antigens in the lymphatic system are saturated with a therapeutic protein of interest, there is greater systemic exposure for the subsequent dose, and thus one can dose less amounts of material or, alternatively, one can dose at longer intervals than might occur otherwise resulting in fewer administrations and use of less therapeutic protein overall.

Preferably, the subcutaneous injection site is chosen to minimize the number of lymphatic glands or nodes accessible to the therapeutic protein prior to entering the thoracic duct, or right lymphatic duct. (The thoracic and right lymphatic ducts are the major ducts which convey lymph into the blood, i.e., the systemic circulation). For example, injection into the upper arm generally targets superficial axillary nodes. The efferent lymph from these nodes then enters the thoracic or right lymphatic duct(s). Subcutaneous injections into the supraclavicular and suprascapular regions may be of greater advantage because lymph from these regions may by-pass the axillary nodes, traversing only the clavicular or scapular nodes on its way to the blood.

Other potentially advantageous injection sites are the abdominal wall and upper thigh. Injection into the abdomen wall may present the monoclonal antibody to superficial lumbar glands and the efferent lymph from these nodes leads to the thoracic duct. Subcutaneous injection in the upper thigh targets superficial inguinal nodes. Efferent lymph from these nodes is eventually received by the lumbar nodes and then passes to the thoracic duct.

It is to be noted that the saturation dose of the instant invention is not equivalent to a loading dose, in which an administered drug, typically an antibiotic, is given iv to rapidly raise the level of antibiotic to its optimum steady state levels. For the instant invention, the kinetics are non-linear and the saturating dose is administered for an entirely different purpose. That is, the initial or saturation dose is intended to bind endogenous target antigens in the lymphatic system in order to allow the second administration of a therapeutic protein, given subcutaneously, to reach the ultimate site(s) of action (e.g., swollen joints in the case of rheumatoid arthritis) and to have its maximum therapeutic effect.

### Disease States

The present invention provides effective treatment for immune cell mediated disease states such as those mediated by T (or B) cells. Such disease states include lymphomas (T and B cell), various leukemias, infectious diseases (e.g., AIDS), transplantation, autoimmune and inflammatory diseases. As a means for inducing immunosuppression, the therapeutic proteins are useful in the treatment, or prophylactic use, of transplanted organ rejection (e.g., heart, lung, kidney, cornea, bone marrow, skin, etc.), for treatment or prevention of autoimmune or inflammatory disease (e.g., rheumatoid arthritis, psoriasis, lupus erythematosus, systemic lupus erythematosus, multiple sclerosis, inflammatory bowel disease, Hashimotos thyroiditis, myasthenia gravis, type I diabetes, uveitis, nephrotic syndrome, atopical dermatitis, etc.), the treatment of reversible obstructive airways disease, intestinal inflammations and allergies (e.g., asthma, Coeliac disease, proctitis, eosinophilic gastroenteritis, mastocytosis, Crohn's disease, ulcerative colitis, etc.), and also food related allergies (e.g., migraine, rhinitis, and eczema).

Preferably, the disease state to be treated is rheumatoid arthritis, asthma, and/or psoriasis. Another preferred embodiment is a means for inducing immunosuppression, such as in the treatment, or prophylactic use, in transplanted organ rejection.

### Dosing

The amount and frequency of sc dosing of a therapeutic protein will depend on the immune cell target antigen (e.g., CD4 receptor), the levels of such target in a patient, the half-life of the therapeutic protein, and the systemic exposure of therapeutic protein needed for effective therapeutic treatment, such that an improvement of disease symptoms is observed. Such parameters can be determined by those of ordinary skill in the art. For example, an effective dose for treating rheumatoid arthritis can be determined by well known means to one of skill in the art such as the assessment of tender joint counts (TJC), swollen joint counts (SJC) [i.e., Ritchie Articular Index, see Ritchie, et al., Q.J. Med., 37:393-406 (1968)], the ACR (American College of Rheumatology) responder index, and/or duration of morning stiffness are but a few means to evaluate effective therapy.

As an example of a dosing regimen, a saturating dose of an anti-CD4 antibody is administered iv in Week 1 at a dose ranging from 80 to 280 mg. Alternatively, the anti-CD4 antibody can be administered twice in Week I (i.e., 40, 80, 100, 120 or 140 mg/bi-weekly). Subsequent dosing is then administered subcutaneously. Such subcutaneous dosing would occur for approximately 3 weeks (see. e.g., Table 5). Repeat dosing would be administered as needed, either when symptoms were observed (i.e., a flare for arthritics) or at a defined interval (e.g., 3, 6 or 9 months). Preferably the dosage is 80-120 mg on a bi-weekly basis. However, one skilled in the art will appreciate that more frequent dosing (e.g., daily, every other day) or less frequent dosing (e.g., weekly) may also be suitable. The amount of therapeutic protein administered can be adjusted accordingly.

In addition, other therapeutic compositions may be coadministered with the therapeutic proteins of this invention. For example, as a treatment of rheumatoid arthritis, DMARDs (disease-modifying antirheumatic drugs) may be coadministered. DMARDs are well known in the art and include methotrexate (mtx), azathioprine, penicillamine, hydroxychloroquine, IM gold, oral gold, sulfasalazine, cyclosporine and chlorambucil.

### Pharmaceutical Composition

Therapeutic proteins of the invention may be prepared as pharmaceutical compositions containing an effective amount of such protein as the active ingredient in a pharmaceutically acceptable carrier. For prophylactic or therapeutic agents of the invention, an aqueous suspension or solution containing the therapeutic protein, such as a monoclonal antibody, preferably buffered at physiological pH, in a form ready for injection is preferred. The compositions for parenteral administration will commonly comprise a solution of an antibody or a cocktail thereof dissolved in an pharmaceutically acceptable carrier, preferably an aqueous carrier. A variety of aqueous carriers may be employed, e.g., 0.4% saline, 0.3% glycine, and the like. These solutions are sterile and generally free of particulate matter. These solutions may be sterilized by conventional, well known sterilization techniques (e.g., filtration). The compositions may contain pharmaceutically acceptable auxiliary substances as required to approximate physiological conditions such as pH adjusting and buffering agents, etc. The concentration of the antibody of the invention in such pharmaceutical formulation can vary widely, i.e., from less than about 0.5%, usually at or at least about 1% to as much as 15 or 20% by weight and will be selected primarily based on fluid volumes, viscosities, etc., according to the particular mode of administration selected.

Non-ionic surfactants suitable for use in the invention preferably have little toxicity to humans and do not cause hemolysis of red blood cells to a significant extent at relevant concentrations. Suitable non-ionic surfactants include, but are not limited to, polysorbates (or polyoxyethylenesorbitans) such as polysorbate 20 (monolaurate), polysorbate 60 (monostearate) and polysorbate 80 (monoloeate). A preferred non-ionic surfactant is polysorbate 80. Polysorbate 80 is generally sold under the trade name of Tween™ 80. The non-ionic surfactant is preferably present in the pharmaceutical composition in the amount of from about 0.01% to about 0.6%, preferably in the amount of about 0.02%.

Sugars useful in the pharmaceutical compositions of the invention serve as bulking agents and tonicity modifiers. Suitable sugars include sugars such as mannitol, sucrose, trehalose and sorbitol. A preferred sugar is sucrose. The sugar can be present in the pharmaceutical composition in an amount of about 3% to about 8% w/w.

In addition to a monoclonal antibody and buffer, the pharmaceutical compositions of the invention may optionally contain other agents suitable for parenteral administration, such as bacteriostatic agents, tonicity modifiers, and cryoprotective agents. Suitable bacteriostatic include benzyl alcohol and methyl and propyl parabens.

A hydrophilic polymeric cryoprotective agent such as hydroxyalkyl cellulose, gelatin, acacia gum, polyvinylpyrrolidone (e.g. molecular weight 10,000 to 60,000) and polyalkylene glycols, such as polyethylene Clycols (e.g. molecular weight 4,000 to 40,000) may be included in the pharmaceutical compositions of the invention. Use of such agent increases stability (that is, minimizes loss of activity and protein degradation) in solution, on lyophilization and upon reconstitution following lyophilization.

The stability of the pharmaceutical composition of the invention is increased at low temperature. Thus, they are preferably stored at temperatures in the range of −70° C. to 15° C., preferably at about −40° C. to about 8° C., more preferably at about 4° C. to about 8° C. Lyophilized compositions are preferably administered within eight hours after reconstitution and are preferably kept at 4° C. to 8° C. after reconstitution.

The pharmaceutical composition of the invention can be contained within a pharmaceutical dosage unit, i.e. a sterile container, such as an ampoule, syringe, vial, bottle or bag, prepared so as to deliver to a patient, especially a human patient, in need thereof an effective amount whether intravenously, subcutaneously, and intramusculary. The precise concentration of mAb in the pharmaceutical dosage unit as well as the precise dose volume of a given dose will depend on such factors as the disease state to be treated, the severity of symptoms and the weight of the patient. Optimization of a given dose of the pharmaceutical compositions of the invention can be carried out in accordance with standard pharmaceutical and medical practice. The concentration of monoclonal antibodies in each pharmaceutical dosage unit can exceed 80 mg/ml. Preferably the concentration of a monoclonal antibody is in the range of about 40 to about 80 mg/ml.

A patient will typically receive a dosage of 1.0 to 4.0 mg/kg/week of mAb. Such treatment would occur for approximately 4 weeks, and then repeat dosing can be administered when necessary (i.e., when a patient exhibits symptoms again (for rheumatoid arthritis, a flare), or at a defined interval thereafter, e.g., 3, 6 or 9 months). For intramuscular administration (i.e., as the initial or saturating dose), the pharmaceutical composition of the invention is administered by injection into a large muscle, such as the anterior thigh. If the total volume of the dose exceed about 5 ml, the dose may be divided into portions and injected into two or more sites.

In a preferred embodiment of the invention, the pharmaceutical compositions are stored in lyophilized form for eventual reconstitution solution such as sterile water or 6% sucrose in sterile water. The lyophilized pharmaceutical compositions are prepared by lyophilizing the aqueous form of the pharmaceutical composition using conventional techniques. The lyophilized pharmaceutical compositions are preferably stored in single dose units for convenience of administration, however, it may be stored in larger quantities in the lyophilized form. The lyophilized composition can be stored in a sterile vial or other container awaiting reconstitution and eventual or immediate parenteral administration. In preparing the pharmaceutical formulation for lyophylization, the aqueous solution can be more dilute than the final reconstituted pharmaceutical compositions. For example, 1 ml of an aqueous solution having 40 mg/ml of mAb is lyophilized, and later reconstituted with 0.5 ml sterile water to provide a solution having 80 mg/ml mAb.

In another preferred embodiment of the invention, the invention is a kit comprising one or more sterile containers of the pharmaceutical composition in lyophilized form and one or more separate sterile containers of solution for reconstitution. The reconstitution solution may also be contained within a different compartment of a multi-compartment container, e.g., a dual compartment syringe designed for convenient mixing and administration. In such syringe or other dual compartment container, the lyophilized mAb and the solution for reconstitution are separated by a membranous barrier which can be ruptured, e.g., by squeezing the syringe or container, thereby mixing the mAb and the solution for reconstitution. The solution for reconstitution, the amount of mAb and the amount of solution for reconstitution in a single kit are selected so as to provide a final reconstituted product having from about 40 to about 80 mg/ml of mAb, at a pH selected in accordance with this invention. A preferred solution for reconstitution is sterile water. The solution for reconstitution may also contain bacteriostatic agents or other substances suitable for parenteral administration.

### T and B Cell Antigens

In addition to the CD4 receptor protein, there are other members of the immunoglobulin superfamily of molecules (Williams et al., Annu. Rev. Immunol., 6:381405 (1988)) which are expressed on T cells, which could also be used as “targets” for therapeutic purposes and administered according to the instant invention. Such molecules include, but are not limited to, CD3, CD8 (CD4 and CD8), CD11/CD18 (see, e.g., Xie et al., J. Immunol., 155: 3619-28 (1995)), CD28 (Aruffo et al., Proc. Natl. Acad. Sci., 84:8573-7 (1987)), CTLA-4, a homologue of CD28 which is expressed transiently at low receptor density on activated CD8+ and CD4+ T cells (Brunet et al., Nature, 328:267-270 (1987)), and gp39 also known as CD40 ligand, T-BAM, and TRAP (see, Noelle et al., Proc. Natl. Acad. Sci., 89: 6550-6554 (1992), Foy et al., J. Exp. Med., 178:1567-1575 (1993) Banchereau et al., Annu. Rev. Immunol., 12:881-922 (1994)). The therapeutic protein can be a monoclonal antibody, or perhaps a soluble ligand (i.e., naturally-occurring ligand free of cytoplasmic and transmembrane domains, optionally fused to the constant region of an immunoglobulin, e.g., Fc region).

Other suitable “target” molecules are the ligands or counter-receptors to CD28, CTLA-4, and gp39, found on B cells. Such ligands or counter-receptors include, CD86 (also known as B7.1) (Linsley et al., Proc. Natl. Acad. Sci., 87: 5031-5035 (1990), Freeman et al., J. Exp. Med., 174: 625-631 (1991)), CD86 (also known as B7.2 and B70) (Freeman et al., J. Exp. Med., 178: 2185-2192 (1993), Freeman et al., Science, 262:909-911 (1993)) and CD40 (Stamenkovic et al., EMBO J., 8:1403 (1989)). With regards to antibodies that bind to CD40, such antibodies should be non-proliferative or non-stimulatory to the B cell (see, e.g., WO94/01547 (Cetus Oncology) and WO95/09653 (Immunex)). Another suitable “target” molecule on human B cells is the CD20 antigen.

The following examples illustrate various aspects of this invention and are not to be construed as limiting in scope. Reagents and materials were obtained from commercial sources unless otherwise indicated.

### EXAMPLES

Materials and Methods

Chemicals.

A macaque/human chimeric antibody that binds human T cell receptor CD4, (mAb CE9.1 (Newman et al., Bio/Technology 10, 1455-1460 (1992)) was used for the following experiments. It is appreciated that other monoclonal antibodies to human CD4 could be used as well. CE9.1 (unlabeled—the reference standard) was supplied as a 5 mg/ml solution or a lyophile with stability enhancing excipients (50 mg/ml upon reconstitution). Soluble CD4 (sCD4, also referred to as sT4, Deen et al., Nature, 331:82-84 (1988)), was obtained as a lyophile (10 mg/ml upon reconstitution). CE9.1 and sCD4 are recombinant proteins expressed in Chinese hamster ovary cells in house. Protein A Sepharose was purchased from Sigma (St. Louis, Mo.). Horseradish peroxidase conjugated mouse anti-human IgG1mAb (clone HP6069) was purchased from Zymed Laboratories Inc. (San Francisco, Calif.). All other chemicals were of reagent grade or better.

[³H]CE9.1.

CE9.1 was metabolically labeled with tritiated leucine in Chinese hamster ovary cells essentially as described in Davis et al. (Drug Metab. Dispos. 20, 695-705 (1992). The antibody was purified from concentrated cultured supernatants by Protein A chromatography (approximately 70% protein recovery overall). Over 400 μCi purified metabolically labeled [³H]CE9.1 was prepared from 5 mCi [³H]leucine (Dupont/NEN, 144 Ci/mmol). The specific radioactivity was approximately 1 μCi/μg (2000 DPM/ng). Pure [³H]CE9.1 was typically greater than 99% trichloroacetic acid precipitable, and 98% of the radiolabel was biologically active with respect to antigen binding (Sepharose-sT4 binding, see below). The radiochemical purity (assessed by SDS-PAGE) typically exceeded 90%.

Animal Husbandry.

Male CD4+ and CD4− transgenic mice (weighing approximately 26 to 43 g) were used for this study and obtained from The Regents of the University of California (Killeen et al., EMBO J. 12, 1547-1553 (1993)). Animals were group-housed (up to 5) in polycarbonate cages with wood chip bedding in a controlled environment (25+2° C.; 50+10% relative humidity) on a twelve hour light/dark cycle. Food (Purina Certified rodent chow, Purina Mills, Inc., St. Louis, Mo.) and filtered tap water were available ad libitum.

1v or sc Administration of [³H]CE9.1.

Animals received an iv dose by injection into a tail vein or a sc dose by injection under the skin in the back. Dose solutions were approximately 0.1 mg/ml [³H]CE9.1 and 100 μCi/ml for the 0.4 mg/kg dose groups. For the 100 mg/kg groups, dose solutions were approximately 14 mg/ml CE9.1 and for the iv group only, 9 μCi/ml. Animals received target doses of 100 μl (low dose) or 200 μl (high dose). The vehicle was a phosphate-glycine buffer or a mixture of this and PBS. The actual dose volume was determined by weighing the syringe before and after dose administration. The actual total radioactivity administered and dose of CE9.1 were determined by analyses of the dose solution (by oxidation/scintillation counting and ELISA, below).

At selected times following drug administration, animals were sacrificed (composite sampling, n=1/timepoint except as noted). In the low dose studies, for the CD4+ iv and sc groups, nominal times of 10, 30, 60, 120, 240, 480 min and 24 and 48 hr were employed. For the CD4− iv and sc groups, nominal times of 10, 120, 480 min and 24 and 48 hr were employed. For the 0.4 mg/kg sc groups (CD4+ and CD4−), additional nominal sacrifice times of 72 hr, 1 and 2 weeks were used. For the high dose studies, for the iv group (CD4+ only), nominal times of 10, 30, 60, 120, 240, 480 min and 24 and 48 hr were employed. For the sc group (CD4+, 100 mg/kg) nominal times of 7, 24, 48, 72 hr and I wk were employed.

Following sacrifice, blood (approximately 1 ml) was removed from the vena cava or by heart puncture, and coagulation was prevented by the addition of 100 μl of 129 mM trisodium citrate. Aliquots of whole blood were placed on combusto-pads for oxidation and the remaining blood was centrifuged to collect plasma. Aliquots of plasma were placed on combusto-pads for oxidation and the remaining plasma and blood cell pellet were frozen on dry ice and stored at approximately −80° C. for further analysis. Liver, kidney, lung, spleen and thymus (or in the high dose iv study, liver and spleen only) were removed to determine total radioactivity. Tissues were either dissolved in ethanolic-KOH, or placed in combusto-cones for direct oxidation. Carcasses were dissolved in ethanolic-KOH to assess total residual radioactivity.

SDS-PAGE.

The plasma and blood radiochemical profile of selected samples was assessed by non-reducing SDS-PAGE. Resolution was accomplished with 8% (w/v) polyacrylamide gels for analysis of plasma protein. Blood cell protein was resolved using 16% (w/v) polyacrylamide gels. To analyze blood cell protein, frozen cells (minus plasma) were thawed at ambient temperature and PBS was added (a volume comparable to the plasma volume withdrawn). Then the cells were resuspended and diluted into SDS loading buffer. Typically, 10-20 μl of plasma or suspended blood cells were loaded directly onto a gel lane for analysis. Following electrophoresis, gels were stained with Coomassie Brilliant Blue R, dried and sliced to assess the radiolabel profile (recoveries >80%). Visual inspection of radiolabel and total protein (Coomassie) profiles, as well as the known electrophoretic mobility of CE9.1 and molecular weight standards were used to assess radiolabel incorporation into endogenous protein (Davis et al., supra).

Sepharose-sT4 Extraction.

To characterize pure radiolabel as well as radiochemical in plasma samples (ex vivo), antigen-binding activity was assessed with a Sepharose-conjugated soluble CD4 (sT4). Plasma samples containing radiolabel (typically <100 μl) were added to a PBS slurry of Sepharose-sT4 (typically 100 μl of 1:1 resin in PBS). In some cases PBS or rat plasma diluted in PBS was also added to facilitate separation of supematant and Sepharose, and to ensure that the total protein concentration was high. After mixing, the supernatant was collected by centrifugation. The percentage of total radiolabel extracted was determined by comparing the radioactivity in an aliquot of the supernatant to the amount of radioactivity in the appropriate aliquot of the original solution. All Sepharose-sT4 binding studies were performed with what was expected to be a large excess of resin relative to the mass equivalents of CE9.1 present.

To estimate the percent administered dose radioactivity in, the radioactivity per volume plasma was scaled to the total radioactivity in total plasma using the weight of the mouse and a literature value for the plasma volume (50 ml/kg, Davies et al., Pharm. Res., 10: 1093-1095 (1993)). To calculate the total radioactivity that bound Sepharose-sT4, the total plasma radioactivity was multiplied by the fraction Sepharose-sT4 binding as assessed above.

ELISA.

Plasma samples were analyzed for CE9.1 concentration using an ELISA based on the simultaneous binding of CE9.1 to antigen (sCD4) and to an anti-human IgGI mAb. In the assay, CE9.1 was captured from plasma in a microtiter plate to which soluble CD4 (sT4) was bound. The sT4/CE9.1 complex then was probed with a C_{H}2 domain specific mouse anti-human IgGl mAb (Hamilton et al., J. Immunol. Methods 158, 107-122 (1993)) conjugated directly to horse-radish peroxidase. Standard curves ranged from 2 to 50 ng/ml CE9.1 in citrated rat plasma or in PBS-containing 10% (v/v) citrated rat plasma (for analysis of samples significantly above the standard curve range). The LLQ (lower limit of quantification) for the sT4/anti-γ1 ELISA was 10 ng/ml in neat mouse plasma (50 μl). Over a concentration range of 10 to 100,000 ng/ml, within-run coefficients of variation ranged from 5.6 to 7.5% while average accuracy ranged from 88 to 110%.

Oxidation of Tritiated Protein for Determination of Radioactivity.

To determine radioactivity in samples containing tritium, liquid aliquots were applied to Combusto-pads in a Combusto-cone. Prior to oxidation, 0.25 mL Combust-Aid was added to the sample. Combustion proceeded in a Packard Model C306 Tri-Carb Sample Oxidizer. Tritiated water was collected into 15-18 mL Monophase S and mixed. For polyacrylamide gel slices, 5 mm×20 mm sections were cut into 2 or 3 pieces and placed directly into a Combusto-cone. Radioactivity in tissues was determined either with liquid aliquots of sample dissolved in ethanolic-KOH, or the tissues were placed in Combusto-cones and oxidized directly. Combustion efficiency (routinely >98%) was assessed by comparing the radioactivity in a combusted and non-combusted standard (Packard Spec-Chec Tritium standard). Prior to scintillation counting, samples were cooled overnight at 4° C. Scintillation counting was performed in a Beckman LS 3800 or 5801 scintillation spectrometer and counting efficiency was determined by the external standard procedure using sealed quench standards.

Pharmacokinetics.

Plasma CE9.1 concentration-time data were analyzed by non-compartmental methods (M. Gibaldi and D. Perrier: “Pharmacokinetics,” 2nd ed. Marcel Dekker, Inc., New York, 1982) using an in-house software package. AUC₀₋ₜ (from the composite ELISA data) was estimated using the log trapezoidal rule for decreasing plasma concentrations and the linear trapezoidal rule for increasing plasma concentrations. The half-life of loss of radioactivity from the spleen (0.4 mg/kg iv, CD4+ mice) was calculated from linear regression of the log-transformed % dose versus time data. Similarly, the reported half-life from the total plasma radioactivity data following sc administration to CD4+ transgenic mice (0.4 mg/kg) was obtained from linear regression of the log-transformed % dose versus time data (from 24 hr to 2 wk).

Intravenous Administration.

Transgenic mice bearing either the hCD4 T cell receptor (CD4+) or no CD4 receptor at all (CD4−, knockouts) received a single low iv dose of metabolically radiolabeled [³H]CE9.1 (0.4 mg/kg) and were sacrificed at selected time points to assess total blood and plasma radioactivity, radioactivity in plasma capable of antigen-binding, and total radioactivity in liver, kidney, lung, spleen, and thymus. Plasma was also analyzed for CE9.1 concentration using the sT4/anti-γ1 ELISA. In a separate study, CD4+ mice received a single high iv dose of [³H]CE9.1 (100 mg/kg) to assess total blood and plasma radioactivity, plasma CE9.1 and total radioactivity in spleen and liver.

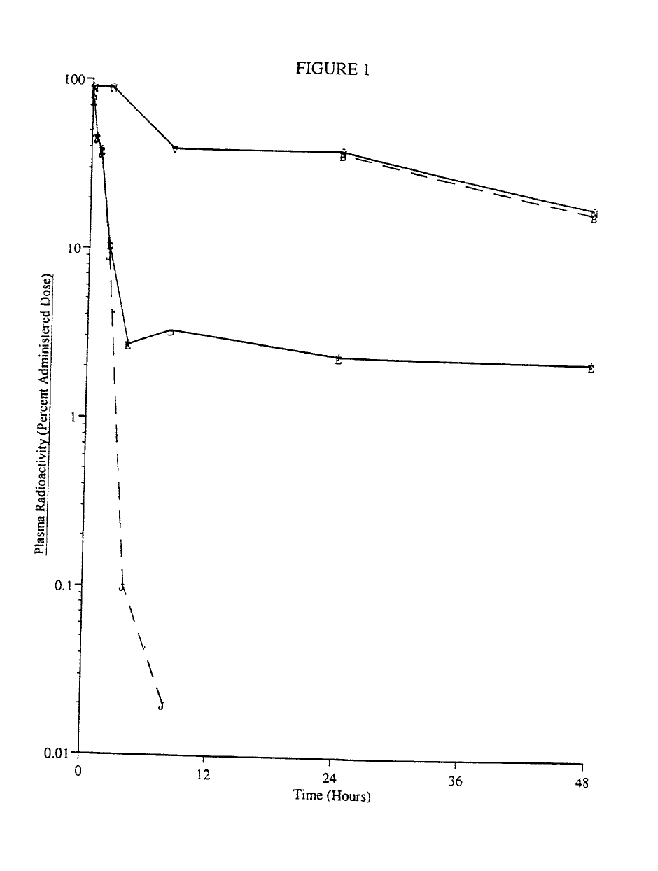

Iv administration of 0.4 mg/kg [³H]CE9.1 to CD4 knockout mice resulted in sustained levels of active antibody in plasma exceeding 19% of the administered radiochemical dose at 48 hr (approximately 1.5 μg/ml by ELISA, FIG. 1). No significant difference was noted between total plasma radioactivity and plasma radioactivity that bound Sepharose-conjugated antigen. Furthermore, blood to plasma ratios to 48 hr did not exceed 0.68 demonstrating insignificant blood cell radiolabel association (Table 2).

By contrast, total percent administered dose radioactivity in plasma fell below 20% less than 2 hr after administration of [³H]CE9.1 to CD4+ transgenic mice (FIG. 1). At 4 hr post-dose, less than 5% of the plasma radioactivity bound Sepharose-conjugated antigen. Plasma CE9.1 concentration was non-quantifiable, by ELISA, 4 hr after iv administration (LLQ of 10 ng/ml).

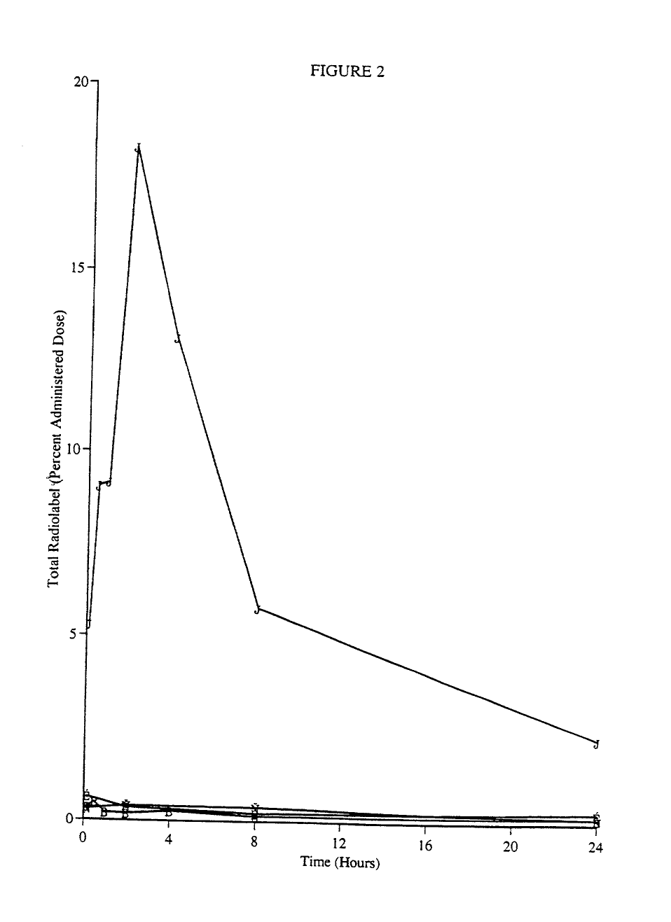

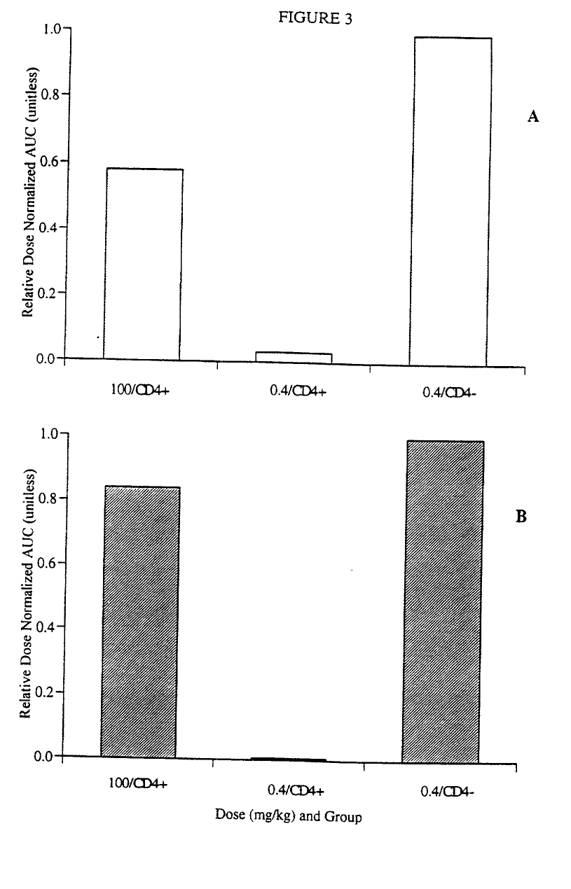

FIGS. 2 and 3 depict total tissue radioactivity versus time following iv administration of [³H]CE9.1 to CD4+ and knockout transgenic mice. Two hr after administration to CD4+ mice, a maximum of approximately 18% of the administered dose was recovered in the spleen, while despite much higher blood levels of active antibody, no more than 0.6% of the administered dose was recovered in the spleen of CD4− mice (FIG. 2). Uptake by the spleen of CD4+ mice occurred in a similar time frame as loss from the plasma compartment. Loss of radiolabel from the spleen of CD4+ mice was characterized by a half-life of approximately 10 hr. Less than 0.5% of the administered dose was recovered in the thymus of CD4+ mice. Only in the liver did total radioactivity approach that of the spleen (maximum of approximately 13%, FIG. 3). In the liver, however, comparable amounts of radiolabel were recovered from knockouts (as much as 9.4%) suggesting that the liver may play a significant role in the disposition of [³H]CE9.1 but perhaps not in an antigen-specific manner. Radioactivity in thymus, kidney and lung together was less than 5.4% in CD4+ or CD4− animals and an average of approximately 45% of the administered radioactivity remained in the carcass after having removed spleen, thymus, liver, kidney and lung.

Administration of a high iv dose of 100 mg/kg [³H]CE9.1 apparently saturated CD4 receptor-dependent aspects of the disposition of the molecule observed at 0.4 mg/kg. In plasma, based on ELISA data, the mean residence time of CE9.1 at 100 mg/kg was approximately 1 day while at 0.4 mg/kg, the mean residence time was less than 1 hr in CD4+ mice. Table 1 shows that the dose normalized AUC (Area under curve)(100 mg/kg, CD4+ mice) was within a factor of 2 of that observed in CD4− animals (0.4 mg/kg). The assumption of pharmacokinetic linearity in a knockout seems reasonable given: 1) the pharmacokinetics should be linear in an animal with no CD4 receptor binding; and 2) the demonstration of pharrnacokinetic linearity of an unrelated IgG1 specific for an exogenous antigen (1-200 mg/kg in monkeys, Davis et al., Drug Metab. Dispos. 23, 1028-1036 (1995)).

Only four hours after iv administration of 0.4 mg/kg [³H]CE9.1, the majority of plasma radioactivity was incorporated into endogenous plasma protein. By contrast, 24 hr after iv administration of 100 mg/kg [³H]CE9.1, plasma radiolabel was primarily intact antibody (80-90%). The blood to plasma ratio (B/P) following iv administration of 100 mg/kg to CD4+ mice is shown in Table 2. CE9.1 partitioned primarily into plasma at all time points to 24 hr following a high iv dose, as it did at low iv doses in CD4− mice. At these time points, radiolabel was primarily intact CE9.1.

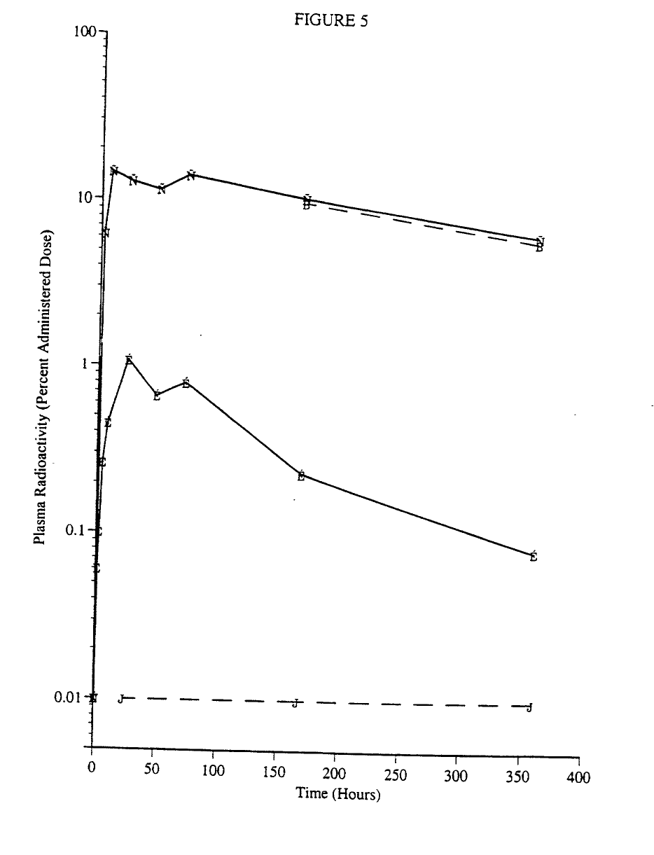

FIGS. 5A and 5B depict the percentage administered dose radioactivity in spleen and liver respectively following iv administration of 100 mg/kg [³H]CE9.1 (CD4+ mice). For comparison, data at 0.4 mg/kg is also shown (CD4+ and CD4− mice). In general, the radiochemical profile in the spleen of CD4+ mice receiving a high iv dose was similar to that observed at the low dose in CD4− mice. At the high dose in CD4+ mice, no clear time dependence of spleen uptake was evident and total administered dose radioactivity was a mean of 0.5%. At low doses, radioactivity reached a maximum value of 18% in 2 hr. By contrast, the profile of percent dose administered radioactivity in the liver was similar in CD4+ mice at 100 mg/kg, CD4+ mice at 0.4 mg/kg and CD4− mice at 0.4 mg/kg; no significant dose or hCD4 receptor-dependence was observed.

Subcutaneous Administration.

CD4+ and CD4− transgenic mice received a single 0.4 mg/kg sc dose of [3H]CE9.1 and were sacrificed at selected time points to assess total blood and plasma radioactivity, radioactivity in plasma capable of antigen-binding, and total radioactivity in liver, kidney, lung, spleen, and thymus. Plasma was also analyzed for CE9.1 concentration using the sT4 (soluble T4 or soluble CD4)/anti-yl ELISA. In a separate study, CD4+ mice received a single 100 mg/kg sc dose of unlabeled CE9.1 to assess plasma anti-hCD4 mAb concentration.

Consistent with the high extravascular systemic exposure of IgG in the absence of endogenous antigen (Davis et al., Drug Metab. Dispos. 23, 1028-1036 (1995)), sc administration of [³H]CE9.1 to CD4 knockout mice resulted in sustained levels of active antibody in plasma exceeding 10% of the administered dose from 8 hr to 1 wk (>1 μg/mL by ELISA, FIG. 5). No significant difference was noted between total plasma radioactivity and plasma radioactivity that bound Sepharose-conjugated antigen as following iv administration to CD4− mice. Furthermore, blood to plasma ratios to 2 wk did not exceed 0.66 demonstrating insignificant blood cell radiolabel association (Table 2).

By contrast, total percent administered dose radioactivity in plasma was at most 1% from 10 min to 2 wk following sc administration of [³H]CE9. I to CD4+ transgenic mice. Plasma radiolabel demonstrated no quantifiable antigen binding activity (FIG. 5). Similarly, plasma CE9.1 concentration was non-quantifiable, by ELISA, throughout the time course (LLQ of 10 ng/ml). Total plasma radioactivity did, however, increase to a maximum value of 1% administered dose at 24 hr and declined with an apparent half-life of approximately 4 days.

Table 1 illustrates relative plasma AUC following iv or Sc administration of [³H]CE9.1 to CD4+ and knockout transgenic mice (0.4 and 100 mg/kg). AUC's were calculated after iv dosing based on a 0-48 hr time interval or after sc dosing on a 0-168 hr time interval. As the dose was increased to 100 mg/kg, CD4 receptor binding was saturated and the resultant AUCs were similar to the AUCs in animals with no receptor. For the 0.4 mg/kg sc data, all plasma concentrations were practically non-quantifiable and the largest possible AUC was estimated based on the assay lower limit of quantification (10 ng/ml).

TABLE 1
Dose normalized plasma AUC in human CD4+ transgenic mice
relative to a 0.4 mg/kg dose in CD4− mice

| Dose | CD4 | | Relative |
|---|---|---|---|
| (mg/kg) | Status | Route | AUC |
| 0.4 | − | iv | 1.0 |
| 0.4 | + | iv | 0.032 |
| 100 | + | iv | 0.58 |
| 0.4 | − | sc | 1.0 |
| 0.4 | + | sc | 0.0062 |
| 100 | + | sc | 0.84 |

For the iv groups, AUCs were calculated based on a 0-48 h time interval whereas for the sc group. AUCs were calculated based on a 0-168 h time interval. For the 0.4 mg/kg CD4+ sc group, all plasma concentrations were nonquantifiable and the largest possible AUC was estimated based on the assay lower limit of quantification. Plasma concentrations of CE9.1 were determined using an ELISA based on soluble CD4 binding

SDS-PAGE analysis of plasma, 4 hr after Sc administration of [³H]CE9.1 to CD4+ transgenic mice, was similar to that following iv administration at the same dose. Blood to plasma ratio at 4 hr was 0.68 indicating that the majority of radiolabel was in plasma at these time points and not associated with blood cells. However, two weeks after administration, the blood to plasma ratio was 7.5 (Table 2). No evidence of cell-bound intact CE9.1 was observed.

Following sc administration, the level of radioactivity in the spleen of CD4+ mice was comparable to that of CD4− mice and very low in comparison to the iv dose group (<0.5% versus a maximum of 18% for CD4+ mice). This was consistent with the fact that analysis of circulating radiolabel failed to detect intact biologically active parent which, based on the 0.4 mg/kg iv data, would be expected to accumulate in the spleen. Of the tissues examined, the liver of CD4+ mice had the highest percentage administered dose radioactivity with at most 2.4%. Similar profiles of liver radioactivity were observed in CD4+ and CD4− mice suggesting that uptake was not CD4 receptor dependent (data not shown).

Given evidence that absorption of antibodies occurs by way of the lymphatics (Weinstein et al., Science 222, 423-426 (1983), Weinstein et al., Cancer Invest. 3, 85-95 (1985), Supersaxo et al., Pharm. Res. 7, 167-169 (1990)), and that the lymphatic system in CD4+ mice contains relatively large amounts of CD4+ T lymphocytes, the lack of systemic availability of [³H]CE9.1 following a low sc dose was likely due to binding to lymphatic CD4+ T cells which prevented antibody from entering the circulation. Furthermore, the presence of radiolabeled endogenous blood and plasma protein following sc administration of [³H]CE9.1 is consistent with the fact that significant metabolism occurred in the lymphatic system.

Following a high sc dose in CD4+ mice, significant absorption of CE9.1 from the injection site into the systemic circulation was observed. Table 1 shows that the dose normalized AUC following a high sc dose to CD4+ mice was within 20% of the AUC that was observed in CD4− mice at low doses. Lymphatic CD4+ T cell binding observed at the low dose was likely saturated at the high dose, and thus CE9.1 was efficiently absorbed into the systemic circulation.

TABLE 2
Blood to plasma ratio of total radioactivity
after iv or sc administration of [³H]CE9.1
to CD4+ and CD4− transgenic mice^{a}

| Route | iv | iv | iv | sc | sc |
|---|---|---|---|---|---|
| Dose | 100 mg/kg | 0.4 mg/kg | 0.4 mg/kg | 0.4 mg/kg | 0.4 mg/kg |
| Group | CD4+ | CD4+ | CD4− | CD4+ | CD4− |
| Time (hr) | | | | | |
| 0.1 | 0.669 | 0.622 | 0.622 | NQ^{b} | NQ |
| 0.5 | 0.660 | 0.639 | ND^{c} | NQ | ND |
| 1 | 0.651 | 0.614 | ND | 0.800 | ND |
| 2 | 0.697 | 0.639 | 0.606 | 0.842 | 0.629 |
| 4 | 0.702 | 0.682 | ND | 0.682 | ND |
| 8 | 0.651 | 0.742 | 0.698 | 0.679 | 0.633 |
| 24 | 0.633 | 1.200 | 0.629^{d} | 0.754 | 0.635 |
| 48 | 0.786 | 1.532 | 0.680 | 1.081 | 0.659 |
| 72 | ND | ND | ND | 1.347 | 0.632 |
| 168 | ND | ND | ND | 2.553 | 0.658 |
| 360 | ND | ND | ND | 7.473 | 0.664 |

^{a}N = 1/timepoint except for 24 and 48 hr iv data where 2 mice were employed (mean value given).
^{b}NQ signifies non-quantifiable (radioactivity in blood and/or plasma too low).
^{c}ND signifies no data (no animal sacrificed at specified timepoint).
^{d}Data from one mouse.

Table 3 illustrates systemic exposure of 2 subcutaneous doses in transgenic mice. In the first instance, the initial dose is saturating, in the later, the initial dose is not saturating.

TABLE 3
Administration of 2 sc doses of CE9.1 to huCD4 mice
when the first dose is saturating
allows the second dose to be absorbed from the injection site
into the systemic circulation
Human CD4 Transgenic Mice

| Dose #1 | Dose #2 | Sacrifice |
|---|---|---|
| t = 0 | t = 7 h | t = 17 h |
| Unlabeled | Radiolabeled | Plasma Radioactivity |
| CE9.1 | CE9.1 | % Dose |
| 100 mg/kg | 0.3 mg/kg | 28* |
| 0.4 mg/kg | 0.3 mg/kg | 1 |

*Radioactivity migrates as intact CE9.1 by SDS-PAGE.

Administration of the initial saturating dose (100 mg/kg) resulted in a 30-fold increase in the radioactivity in plasma 17 hours after administration of the second dose.

In summary, the disposition of the anti-hCD4 mAb was shown to be highly dependent on the presence and distribution of the human CD4 receptor. After a low iv dose, rapid loss of [³H]CE9.1 from the plasma compartment was accompanied by accumulation of radioactivity in the spleen. By contrast, in CD4− mice, CE9.1 had a long plasma half-life as expected for an IgG1 in the absence of endogenous antigen. These phenomenon were dose dependent. Saturation of CD4 receptor binding following a high iv dose resulted in pharmacokinetics and distribution consistent with that observed at lower doses in animals with no CD4 receptor.

After a low sc dose, no evidence of absorption of intact, active CE9.1 from the injection site into the systemic circulation was observed. However, high systemic exposure was noted following sc administration of the same dose to CD4− mice suggesting that binding of CE9.1 to the CD4 receptor prevented the mAb from entering the systemic circulation. Again, these phenomenon were dose dependent. Saturation of CD4 receptor binding following a high sc dose resulted in pharmacokinetics consistent with that observed at lower doses in animals with no CD4 receptor.

Comparative data of CE9.1 in transgenic mice and man suggest that the intravenous pharmacokinetics are qualitatively similar in these species and thus the discoveries described herein in the mouse should be informative about the disposition of CE9.1 in man. Specifically, in both the transgenic mouse and man, the pharmacokinetics are non-linear. Greater than dose proportional increases in the area under the plasma concentration versus time curve are observed as the intravenous dose is increased in both the transgenic mouse and man. This non-linearity is likely due to movement of CD4 positive T cells in and out of the peripheral blood compartment (trafficking) and saturable binding of CE9.1 to CD4. The CD4 antigen has a similarly important role in dictating the behavior of CE9.1 in vivo in both transgenic mouse and man.

It has previously been demonstrated in these CD4 transgenic mice, that the human CD4 gene restores normal helper cell functions to mice with no endogenous CD4 gene (Killeen et al., EMBO J. 12, 1547-1553 (1993)). Furthermore, immunohistochemical studies of the distribution of CD4 in the CD4+ transgenic mouse show that the human CD4 molecule is expressed on T lymphocytes and cells of macrophage/dendritic cell lineage similar to the reported distribution in humans. Together these data suggest that the transgenic mouse model will be predictive of the behavior of CE9.1 in man and that the data described in the present invention are important in optimizing clinical dosing regimens to maximize therapeutic benefit.

Chronic Dosing Regimen for Anti-CD4 Monoclonal Antibody

Anti-CD4 monoclonal antibody is currently administered via intravenous administration for treatment of T-cell mediated autoimmune diseases such as rheumatoid arthritis and asthma. A typical dosing regimen is presented in Table 4.

For chronic subcutaneous dosing, a suitable dosing regimen would comprise the regimen outlined in Table 5.

The present invention has been described with reference to specific embodiments. However, this application is intended to cover those changes and substitutions which may be made by those skilled in the art without departing from the spirit and the scope of the appended claims.

TABLE 4
Potential Chronic Dosing Regimens for Anti-CD4 Monoclonal Antibody in Rheumatoid Arthritis
and Asthma Intravenous administration only
Protocol A

| Week 1 | | Week 2 | | Week 3 | | Week 4 | |
|---|---|---|---|---|---|---|---|
| Monday | Thursday | Monday | Thursday | Monday | Thursday | Monday | Thursday |
| 80 mg iv | 80 mg iv | 80 mg iv | 80 mg iv | 80 mg iv | 80 mg iv | 80 mg iv | 80 mg iv |

Protocol B

| Week 1 | Week 2 | Week 3 | Week 4 |
|---|---|---|---|
| Monday | Monday | Monday | Monday |
| 160 mg iv | 160 mg iv | 160 mg iv | 160 mg iv |

TABLE 5
Regimen 1 - Intravenous administration initially to saturate the lymphatic system, followed by
chronic subcataneous* dosing

| Week 1 | | Week 2 | | Week 3 | | Week 4 | |
|---|---|---|---|---|---|---|---|
| Monday | Thursday | Monday | Thursday | Monday | Thursday | Monday | Thursday |
| 80 mg iv** | 80 mg sc | 80 mg sc | 80 mg sc | 80 mg sc | 80 mg sc | 80 mg sc | 80 mg sc |

Regimen 2 - Subcataneous administration initially to saturate the lymphatic system, followed by
chronic subcataneous dosing

| Week 1 | | Week 2 | | Week 3 | | Week 4 | |
|---|---|---|---|---|---|---|---|
| Monday | Thursday | Monday | Thursday | Monday | Thursday | Monday | Thursday |
| 80 mg sc | 80 mg sc | 80 mg sc | 80 mg sc | 80 mg sc | 80 mg sc | 80 mg sc | 80 mg sc |

Regimen 3 - As in Regimen 2 with weekly dosing 160 mg/dose

| Week 1 | Week 2 | Week 3 | Week 4 |
|---|---|---|---|
| Monday | Monday | Monday | Monday |
| 160 mg iv** | 160 mg sc | 160 mg sc | 160 mg sc |

*Subcataneous dosing interval and dose level may not necessarily be restricted to that employed following iv administration.
**Initial dose may also include im administration.

What is claimed is:

1. An improved method for treating immune cell mediated diseases wherein the improvement comprises: administering a saturating dose of a therapeutic protein selected from the group consisting of a monoclonal antibody, a soluble receptor and a soluble ligand which binds to an antigen expressed on the surface of an immune cell; followed by a second administration of said therapeutic protein, wherein the second administration is given subcutaneously, and wherein the systemic exposure of said therapeutic protein from the second administration is at least 50% greater than the systemic exposure from a first, and equivalent, subcutaneous dose of the therapeutic protein.

2. The method of claim 1 wherein the immune cell is a T-cell lymphocyte.

3. The method of claim 2 wherein the T-cell antigen is human CD4.

4. The method of claim 2 wherein the T-cell antigen is human CD28.

5. The method of claim 2 wherein the T-cell antigen is human CTLA-4.

6. The method of claim 2 wherein the T-cell antigen is human CD40 ligand.

7. The method of claim 1 where in the monoclonal antibody is a primate-human chimeric antibody.

8. The method of claim 7 wherein the chimeric antibody is CE9.1.

9. The method of claim 1 where in the monoclonal antibody is a humanized monoclonal antibody.

10. The method of claim 1 where in the monoclonal antibody is a human monoclonal antibody.

11. The method of claim 2 wherein the T-cell mediated disease is rheumatoid arthritis.

12. The method of claim 2 wherein the T-cell mediated disease is psoriasis.

13. The method of claim 2 wherein the T-cell mediated disease is asthma.

14. The method of claim 2 wherein the T-cell mediated disease is graft verses host disease.

15. The method of claim 1 wherein the saturating dose is given intravenously.

16. The method of claim 1 wherein the saturating dose is given intramuscularly.

17. The method of claim 1 wherein the second administration of said therapeutic protein is given subcutaneously in the upper arm, the supraclavicular or suprascapular region.

18. The method of claim 1 wherein the second administration of said therapeutic protein is given subcutaneously in the abdominal wall or upper thigh.

19. The method of claim 1 wherein the immune cell is a B-cell lymphocyte.

20. The method of claim 19 wherein the B-cell antigen is human CD40.

21. The method of claim 19 wherein the B-cell antigen is human CD80.

22. The method of claim 19 wherein the B-cell antigen is human CD86.

23. The method of claim 19 wherein the B-cell antigen is human CD20.

24. The method of claim 19 wherein the therapeutic protein is a monoclonal antibody to human CD80 or CD86.

25. The method of claim 19 wherein the therapeutic protein is a monoclonal antibody to human CD20.

26. The method of claim 19 wherein the therapeutic protein is a non-proliferative monoclonal antibody to human CD40.

27. The method of claim 19 wherein the B-cell mediated disease is B-cell lymphoma.

28. The method of claim 19 wherein the B-cell mediated disease is rheumatoid arthritis.

29. The method of claim 19 wherein the B-cell mediated disease is psoriasis.

30. The method of claim 19 wherein the B-cell mediated disease is asthma.

31. The method of claim 19 wherein the B-cell mediated disease is graft verses host disease.

32. The method of claim 1 wherein the systemic exposure of said therapeutic protein from the second administration is at least 2-fold (i.e., 100%) greater than the systemic exposure from a first, and equivalent, subcutaneous dose of the therapeutic protein.

33. The method of claim 1 wherein the systemic exposure of said therapeutic protein from the second administration is at least 4-fold greater than the systemic exposure from a first, and equivalent, subcutaneous dose of the therapeutic protein.
